(19) United States
(12) Patent Application Publication
(10) Pub. No.: US 2019/0099135 A1
(43) Pub. Date: Apr. 4, 2019

(54) Band Strap Fastening System for Imaging Equipment Tables

(71) Applicant: Columbus Healthcare Products, LLC, Columbus, OH (US)

(72) Inventor: Janet M. Kellogg, Columbus, OH (US)

(21) Appl. No.: 16/146,286

(22) Filed: Sep. 28, 2018

Related U.S. Application Data
(60) Provisional application No. 62/565,554, filed on Sep. 29, 2017.

(51) Int. Cl. A61B 6/04; A44B 13/00; A61B 5/055

(52) U.S. Cl. CPC A61B 6/0421; A61B 5/0555; A44B 13/00

(57) ABSTRACT

A fastening apparatus for an imaging device having a table upon which a patient may rest. A first track portion has spaced lips defining a channel with a first longitudinal axis. The lips are separated at their terminal ends to define an elongated slot. A second track portion includes a panel attached to a rod. The rod has a second longitudinal axis and may be disposed in the channel of the first track portion with the first and second longitudinal axes substantially parallel and the panel disposed in the slot. The first track portion is thus longitudinally movable relative to the second track portion. One of the track portions may attach to the table and the other track portion may attach to a band for extending around the patient.

### CROSS-REFERENCES TO RELATED APPLICATIONS

This application claims the benefit of U.S. Provisional Application No. 62/565,554 filed Sep. 29, 2017. The prior application is hereby incorporated by reference.

### STATEMENT REGARDING FEDERALLY-SPONSORED RESEARCH AND DEVELOPMENT

(Not Applicable)

### THE NAMES OF THE PARTIES TO A JOINT RESEARCH AGREEMENT

(Not Applicable)

### REFERENCE TO AN APPENDIX

(Not Applicable)

### BACKGROUND OF THE INVENTION

The invention relates generally to imaging equipment, and more particularly to an attachment system for bands or straps used on patients resting on imaging equipment tables.

In conventional x-ray, MRI and other imaging equipment, patients are placed on tables prior to the procedure being initiated. During the subsequent procedure, the patient must remain essentially motionless so the resulting image of the patient's body is clear across the entire image. A clear image permits those who read the image to best diagnose the condition of the imaged portion of the patient's anatomy.

Imaging equipment tables from an original equipment manufacturer (OEM) have bands and other devices that assist patients in remaining motionless. Such bands extend over the patient from the table on opposite sides of the patient and fasten to one another. Usually there is one band on one side of the patient and another complementary band on the opposite lateral side. Bands can be narrow, in the manner of an automobile seatbelt, or wide, in the manner of a torso-width panel of flexible, but not substantially elastic, fabric.

Some bands are used with a system that is integral to the table. However, replacement bands must then be obtained from the manufacturer of the table and attached in place of used bands. These are expensive and omit many advantages of aftermarket bands. Additionally, many OEM tables have no such system, but have removable fasteners, such as longitudinally-mounted strips of VELCRO® brand hooks and loops fasteners, mounted to the tops of the tables.

Whether OEM or aftermarket, all bands must be cleaned after every use. OEM bands may be adjustable in where, along the length of the table, they fasten, but aftermarket bands are not known to be adjustable. Aftermarket bands may be cleaned and replaced quickly. However, such aftermarket band systems may be visible in the resulting image due to being made of radiopaque (opaque to x-rays or similar radiation) materials, thereby introducing possible errors in the diagnosis.

### BRIEF SUMMARY OF THE INVENTION

Disclosed herein is a system for removably attaching bands to imaging equipment tables. The disclosed system permits longitudinal adjustment of the bands relative to the table, including longitudinal movement of the bands and the patient after the patient is positioned on the table. Longitudinal adjustment after patient placement is advantageous, because patients are often imaged to assess an injury, and longitudinal adjustability avoids or mitigates further injury and the loss of time that would otherwise be spent removing the band prior to adjusting the patient's position and replacing the band after adjusting.

The term “longitudinal” refers to a direction or orientation along the length of the table, so “longitudinal adjustment” refers to adjustment by moving along the length of the table. The imaging device may be any conventional equipment used to take images of a human or animal body while the patient lies on the table thereof. Common examples of imaging devices are x-ray and MRI machines, but there are many others. Typical tables of such machines are 6-7 feet long and about 18 to 36 inches wide, although the sizes may vary depending upon the intended use of the equipment.

In one embodiment, a first track portion may be removably or permanently mounted to the side or top of a table of an imaging device, and a second track portion may be mounted to the edge of a band that will extend across the patient's body. A similar or identical pair of track portions may be attached at the opposite lateral side of the table. The pairs of track portions are shaped as complements to one another so that the first and second track portions may fasten together to permit the attached band to extend laterally over a patient's body and hold the patient firmly in the lateral direction. The track portions prevent or limit the first and second track portions from moving appreciably relative to one another in the direction along which tension is applied by the band to the track portions. This direction is generally referred to as the “lateral” direction, which is transverse, and preferably perpendicular, to the longitudinal direction. The track portion pair on the opposite side of the table cooperates similarly so that when a band from each track portion pair extends across the patient and is fastened, the track portions attached to the bands do not permit substantial lateral movement of the patient.

The track portions retain the ability to displace the bands longitudinally relative to the table even while restricting the patient in the lateral direction. The first track portion and the second track portion may be mounted to one another by a rod aligned in a channel where the rod is similar enough in size to the channel to be incapable of removal through a slot formed along the length of the channel, but the rod may be removed by sufficient movement in a longitudinal direction.

Preferably one pair of track portions is mounted to the table near one side, and a similar pair of track portions is mounted to the table on an opposite side. The pairs of track portions are preferably parallel to one another. In a preferred embodiment, the track portions that mount to the table have fasteners on the table-facing sides that fasten to the table's fastening system. On the opposite sides of the track portions that face away from the table, secondary fasteners are formed so that any structure that is designed to attach to the fasteners that the track portions attach to may attach to these secondary fasteners.

The track portions and the band may be “radiolucent.” A radiolucent structure is one that, when a conventional x-ray (or other imaging, such as MRI, CT and Gamma camera) image is taken in a conventional manner, and in which the radiolucent structure is in the field of view, the radiolucent structure is not visible on the developed image, or at least is so insubstantially visible that it does not interfere with the diagnosis. Thus, the term “radiolucent” is similar to “translucent,” except that the former relates to images using high energy radiation rather than visible light as in the latter.

A first track portion mounted to the table is formed to accept an opposing interlocking second track portion which attaches to a band. A second pair of track portions is mounted on the opposite side of the table. The system may be retrofitted (after manufacture and sale to the end user, but possibly before) to existing tables where the track and attachment means extend along the side of the table. Thus, both the track and the band may be completely removable from the table, if desired, affording flexibility. The band can slide along the side of the table using the track and the interlocking connection, thereby allowing the band to be repositioned while the patient is already on the table.

Disclosed herein is a fastening apparatus for an imaging device. The imaging device has a table upon which at least part of a patient may rest while an image is taken of the patient. The apparatus comprises a first track portion that has spaced lips defining a channel with a first longitudinal axis. The lips are separated at their terminal ends to define an elongated slot. A second track portion includes a panel attached to a rod. The rod has a second longitudinal axis and a size that permits the rod to be disposed in the channel of the first track portion with the first and second longitudinal axes substantially parallel and the panel disposed in the slot. In this configuration, the first track portion is longitudinally movable relative to the second track portion. Further, one of the first and second track portions is configured for attachment to the table and another of the first and second track portions is configured for attachment to a first band that may extend around the patient.

In a preferred embodiment, the fastening apparatus further comprises a third track portion having spaced lips defining a channel therebetween and a third longitudinal axis. The lips are separated at their terminal ends to define a slot. A fourth track portion has a panel attached to a rod. The rod has a fourth longitudinal axis and a size that permits it to be disposed in the channel of the third track portion with the third and fourth longitudinal axes substantially parallel and the panel disposed in the slot. In this configuration, the third track portion is longitudinally movable relative to the fourth track portion. Further, one of the third and fourth track portions is configured for attachment to the table and another of the third and fourth track portions is configured for attachment to a second band that is attachable to the first band.

In one embodiment, the first track portion is configured for attachment to a first side of the table and the third track portion is configured for attachment to a second, opposite lateral side of the table. The second track portion is configured for attachment to the first band and the fourth track portion is configured for attachment to the second band.

In another embodiment, the second track portion is configured for attachment to a first side of the table and the fourth track portion is configured for attachment to a second, opposite lateral side of the table. The first track portion is configured for attachment to the first band and the third track portion is configured for attachment to the second band.

Disclosed herein is an imaging apparatus having a table upon which at least part of a patient rests while an image is taken of the patient. The imaging apparatus comprises a first track portion that has spaced lips defining a channel with a first longitudinal axis. The lips are separated at their terminal ends to define an elongated slot. A second track portion has a panel that is attached to a rod having a second longitudinal axis. The rod is disposed in the channel of the first track portion with the first and second longitudinal axes substantially parallel and the panel disposed in the slot. In this configuration, the first track portion is longitudinally movable relative to the second track portion. Further, one of the first and second track portions is mounted to the table and another of the first and second track portions is mounted to a first band that extends at least partially across the patient while the image is taken.

In a preferred embodiment, the imaging apparatus further comprises a third track portion having spaced lips defining a channel with a third longitudinal axis. The lips are separated at their terminal ends to define an elongated slot. A fourth track portion has a panel attached to a rod with a fourth longitudinal axis. The rod is disposed in the channel of the first track portion with the third and fourth longitudinal axes substantially parallel and the panel disposed in the slot. In this configuration, the third track portion is longitudinally movable relative to the fourth track portion. Further, one of the third and fourth track portions is mounted to the table and another of the third and fourth track portions is mounted to a second band that is attached to the first band.

In one embodiment, the first track portion is mounted to a first side of the table and the third track portion is mounted to a second, opposite lateral side of the table. The second track portion is mounted to the first band and the fourth track portion is mounted to the second band.

In another embodiment, the second track portion is mounted to a first side of the table and the fourth track portion is mounted to a second, opposite lateral side of the table. The first track portion is mounted to the first band and the third track portion is mounted to the second band.

Disclosed herein is a method of taking an image of at least part of a patient while the patient rests upon a table. The method comprises a step of extending a first band over the at least part of the patient. The first band is mounted at one end to one of first and second track portions. Another of the first and second track portions is mounted to the table. More specifically, the first track portion has spaced lips defining a channel with a first longitudinal axis. The lips are separated at their terminal ends to define an elongated slot. The second track portion has a panel attached to a rod with a second longitudinal axis. The rod is disposed in the channel of the first track portion with the first and second longitudinal axes substantially parallel and the panel disposed in the slot. The method includes a step of tightening the first band around at least part of the patient by applying to the first band a tensile force that is transverse to the first and second longitudinal axes. The method also includes a step of displacing at least the band relative to the table along the first and second longitudinal axes.

In a preferred embodiment, the method further comprises the step of attaching a second band to the first band. The second band is mounted at one end to one of the third and fourth track portions and another of the third and fourth track portions is mounted to the table. More specifically, the third track portion has spaced lips defining a channel with a third longitudinal axis. The lips are separated at their terminal ends to define an elongated slot. The fourth track portion has a panel attached to a rod with a fourth longitudinal axis. The rod is disposed in the channel of the third track portion with the third and fourth longitudinal axes substantially parallel and the panel disposed in the slot. This method includes another step of applying to the second band a tensile force that is transverse to the third and fourth longitudinal axes.

The embodiments permit easily longitudinal adjustment to band placement along an imaging table, which permits a patient to be moved longitudinally after the bands have been fastened over the patient and a transverse, lateral force it applied. One embodiment includes a track system attached to the cradle under the table pad with hook and loop fasteners that are complementary to OEM hook and loop fasteners fastened on the table. This device is compatible with CT, MRI, NM and RO imaging equipment and does not negatively interfere with the same. The track system is made of materials that are strong enough to limit patient movement during imaging. In some embodiments, the track system may be removed after imaging for cleaning or disposal.

### BRIEF DESCRIPTION OF THE SEVERAL VIEWS OF THE DRAWINGS

FIG. 1 is a view in perspective illustrating a preferred track portion with a channel.

FIG. 2 is a view in perspective illustrating a preferred track portion with a rod.

FIG. 3 is an end view illustrating the track portion of FIG. 1.

FIG. 4 is a view in perspective illustrating a conventional imagining equipment table with an embodiment of the present invention mounted in an operable position.

FIG. 5 is a view in perspective illustrating a conventional imaging equipment table with an embodiment of the present invention mounted in an operable position.

FIG. 6 is a view in perspective illustrating an embodiment of a band with a track portion mounted thereto, and another track portion mounted in an operable position on the band.

FIG. 7 is a front view illustrating an embodiment of a track portion.

FIG. 8 is a rear view illustrating the embodiment of FIG. 7.

FIG. 9 is a view in perspective illustrating a band with a track portion mounted thereto, and another track portion mounted in an operable position on the band.

FIG. 10 is a view in perspective illustrating another embodiment.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

U.S. Provisional Application No. 62/565,554 filed Sep. 29, 2017, is hereby incorporated by reference.

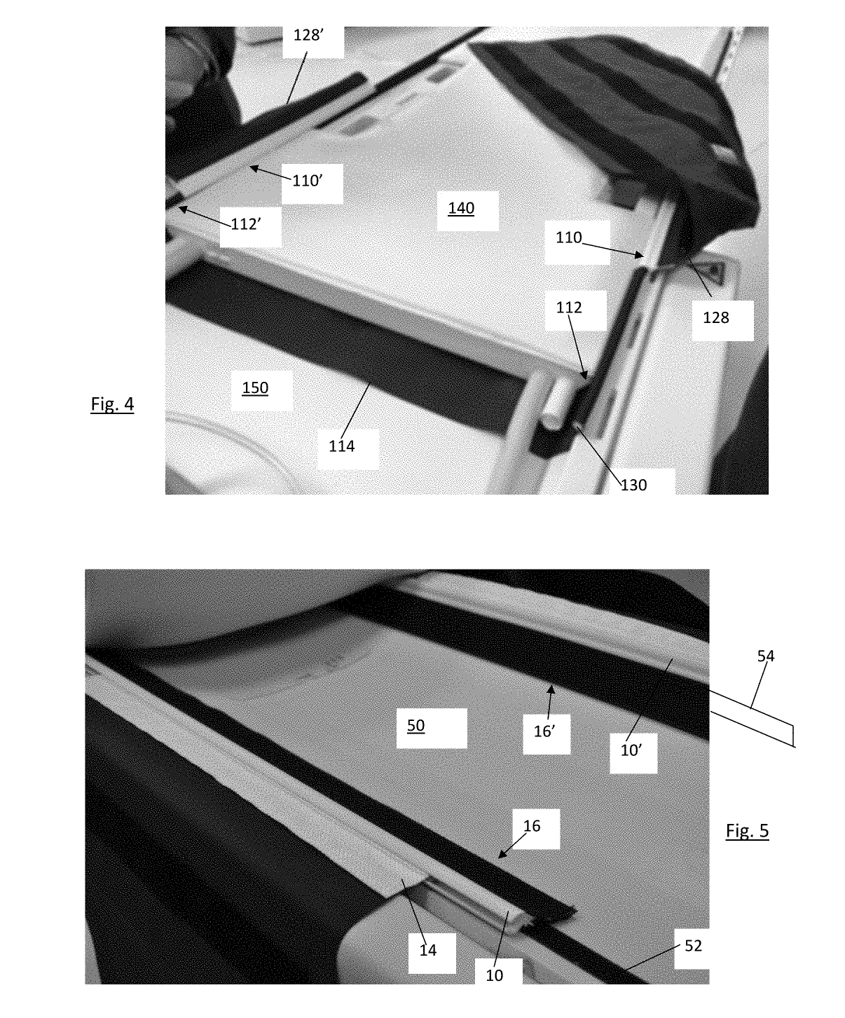

On conventional imaging equipment tables, examples of which are shown in FIGS. 4 and 5, substantially parallel hooks and loops fastening strips may be mounted near opposing edges of the table. The table 50 has example strips 52 and 54, which are adhesively or otherwise mounted to the table 50 substantially parallel to one another with the hooks or loops sides facing upwardly away from the table so they may be contacted by the complementary loops or hooks sides of corresponding strips, as described below. It is contemplated that other fasteners may be used in place of hooks and loops strips, including without limitation, snaps, screws, buttons, adhesives, welding, or any other fasteners that are conventionally, or are in the future, attached to such imaging equipment tables. A person having ordinary skill will understand how to attach the structures described herein using fasteners other than hooks and loops strips.

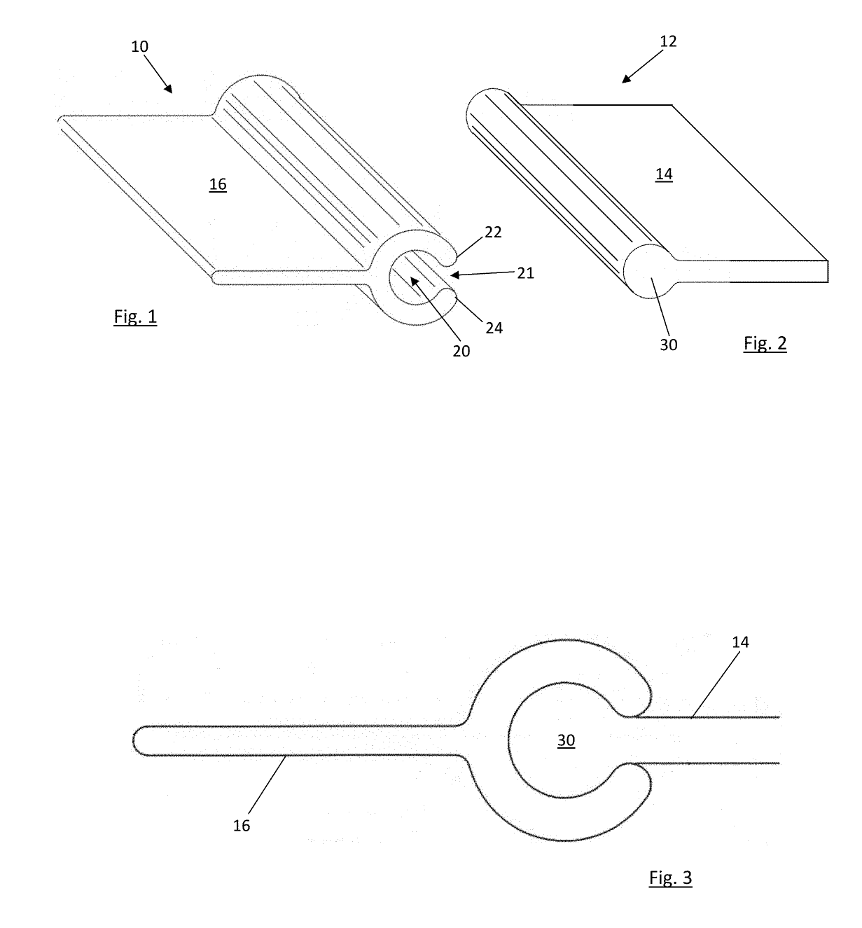

In one embodiment, the track portions 10 and 12 (see FIGS. 1-3) may be attached to fasteners on a table and/or a band, as described in more detail herein. The track portion 10 may be mounted to the table by mounting a panel 16 that is integral with the track portion 10 to fasteners that are conventionally provided on the top of the table. In one embodiment, a strip of hooks and loops fastener is mounted to the panel 16, such as by sewing, adhesive or any other means, and then the combination of the panel 16 and attached strip of hooks and loops fastener is pressed against the complementary strip 52, as shown in FIG. 5. A similar track portion 10′ may have a similar panel 16′ with a strip of hooks and loops fasteners mounted thereto and may be pressed against the complementary strip 54.

The track portion 10 has a channel 20 defined by the opposing and separated lips 22 and 24. The channel 20 may be cylindrical in shape, with a longitudinal axis, but this is not critical. A longitudinal and elongated slot 21 is defined by the space between the terminal ends of the lips 22 and 24 and extends the length of the track portions 10 and 10′.

The track portion 12 may be attached to the track portion 10 by various means. In one embodiment the track portion 12 has an enlarged edge that may be formed by a cylindrical rod 30 sewn, adhered, welded, otherwise fixed to, or integrally formed in, the edge of the track portion 12. In one embodiment, the rod 30 is a polymer cording that is surrounded by flexible fabric that is sewn, adhered or otherwise mounted to the panel 14 of the track portion 12. The cording may be 7.5 mm in diameter, but other sizes are contemplated. It is also contemplated that the track portion 12, including the rod 30, may be cast, extruded or otherwise integrally formed of one or more materials.

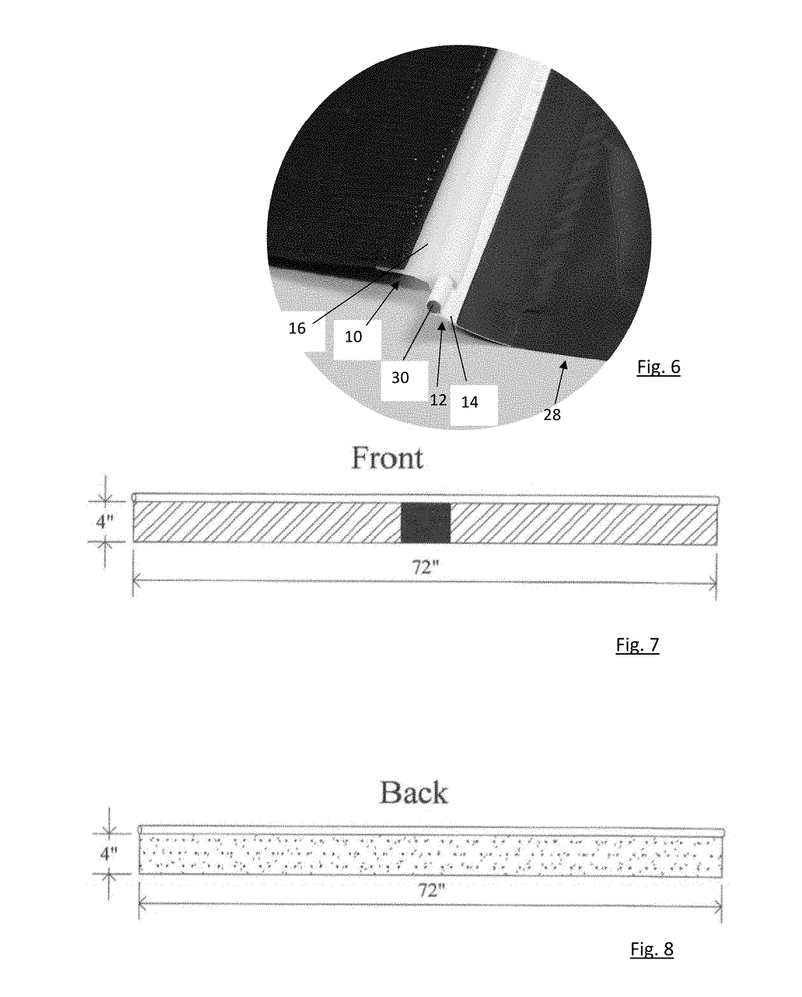

The enlarged part of the track portion 12 formed by the rod 30 engages the track portion 10 as shown in FIGS. 3 and 6, such as by the rod 30 being inserted coaxially into the channel 20 inside the two lips 22 and 24 of the track portion 10. The rod 30 is preferably a diameter that fits within the channel 20 that is formed between the opposing lips 22 and 24 of the track portion 10 without substantial compression of the rod 30. The channel 20 may be substantially cylindrically-shaped, particularly if the rod 30 is also cylindrically-shaped. The rod 30 has a longitudinal axis, and when the rod 30 is inserted into the channel 20, the longitudinal axis of the rod 30 and the longitudinal axis of the channel 20 may be parallel. In some embodiments the axes may be coaxial.

The rod 30 has a diameter and physical characteristics, such as low compressibility in typical conditions, that prevent it from being removed from the channel 20 except by being moved along the length of the channel 20 to be withdrawn from the channel 20 at an end of the track portion 10. In one embodiment, movement of the rod 30 perpendicular to the axis of the channel 20 is mechanically prohibited, such as by the slot 21 being narrower than the diameter of the rod 30 and the rod 30 being insufficiently compressible under typical conditions. The slot 21 may be at least as wide as the thickness of the panel 14, is preferably substantially narrower than the diameter of the rod, and may be about one-third of the diameter of the rod 30. Thus, if the material of which the track portion 10 is made is substantially strong, and the rod 30 is not substantially compressible, the rod 30 may not be removed from, or inserted into, the channel 20 via the slot 21. Instead, the rod 30 may be inserted into the channel 20 from a longitudinal end only, and the track portion 12 may be moved only longitudinally relative to the track portion 10. Removal is accomplished by sliding the track portion 12 so that the rod 30 moves along the length of the channel 20 to the end until the entire rod 30 is removed from the channel 20.

The track portions 10 and 12 may be made of plastic, metal, composite or other suitable materials. The track portions may be made from High density Polyethylene (HDPE) that is radiolucent when using MRI, CT and Gamma cameras.

In the configuration described above the track portions 10 and 10′ are securely fastened to the table 50 in a manner understood by the person of ordinary skill. Preferably, the respective longitudinal channels of the first and second track portions 10 and 10′ face outwardly away from the longitudinal axis of the table as shown in FIG. 5, but this is not critical.

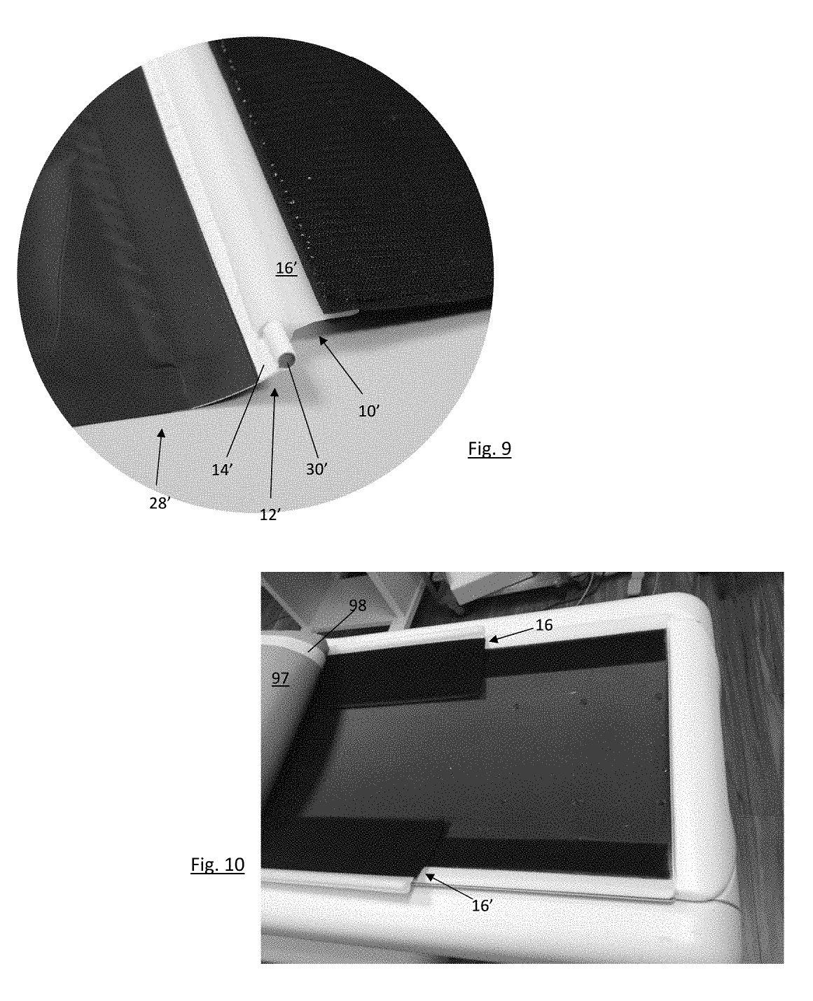

A band, such as the band 28 shown in FIG. 6, may be mounted to the panel 14 of the track portion 12, such as by welding (e.g., RF welding). A similar band 28′ (FIG. 9) may be mounted to the panel 14 of the track portion 12′. Bands used with the invention may be narrow (about 1 to about 4 inches) or wide (about 5 to about 24 or more inches). The width of the bands is measured in the longitudinal direction, which is from one end of the table to the opposite end. Thus, a six inch wide band measures six inches from the edge of the band closest to the head of a human patient placed on the table to the edge of the band closest to the foot of the patient. The dimensions of the band may affect the dimensions of the track portions, because the track portions may be the same length as the bands. This is apparent from at least FIGS. 6 and 9.

The band 28 attached to the track portion 12 and the band 28′ attached to the track portion 12′ may be fixed to one another by fasteners, which may be hooks and loops fasteners. This is common with conventional imaging table bands, because such bands are typically fixed to one another in the manner of a belt, such as by hooks and loops fasteners mounted to facing surfaces of the bands, or by other fasteners. Once the bands 28 and 28′ have been extended from opposite sides over the patient's body in an overlapping relationship, they are placed into contact with one another in that overlapping relationship and tightened against the patient's body. For example, the band 28 may be on the left side of a patient reclined on the table 50 and the band 28′ may be on the right side of the patient. The band 28 may be extended over the patient's torso to rest thereupon. The band 28′ may then be extended over the patient's torso and that portion of the band 28 that is resting on the torso. The bands 28 and 28′ may have complementary hooks and loops fastening strips mounted to the facing surfaces so that the two bands become fixed in a conventional manner when the bands are overlapped and the fastening strips are brought into contact with one another. This fixing process is preferably performed while both bands 28 and 28′ are pulled in tension across the patient's torso. Once thus fastened, the bands 28 and 28′ do not permit substantial lateral movement of the patient unless the patient deliberately moves.

When the bands 28 and 28′, which are mounted with the rods 30 and 30′ in the channels of the track portions 10 and 10′, respectively, are pulled laterally across the patient's body, the tensile force on the bands are transverse, and may be perpendicular, to the track portions 10 and 10′. Under normal use, this lateral force is insufficient to remove the rods 30 and 30′ from the channels in the respective the track portions 10 and 10′ in a transverse direction, i.e., through the slots. However, the track portions 12 and 12′, along with the bands 28 and 28′, may be displaced longitudinally relative to the table. This is due to the ability of the rods 30 and 30′ to move longitudinally relative to the channels in the track portions 10 and 10′. The relative sizes of the rods and channels permits relative longitudinal sliding between the rods 30 and 30′ and the lip structures defining the channels in the track portions 10 and 10′. This permits movement of the patient and the bands even after the patient has been placed on the table and the bands are tightened over the patient. Movement is accomplished by simply applying a longitudinal force to the patient, the bands or both until the force is sufficient to cause relative movement. All the while, the rods 30 and 30′ are not removed from the channels of the track portions 10 and 10′ through the slots due to lateral forces.

As shown in FIGS. 6 and 9, the sides of the panels 16 and 16′ that face toward the table have fasteners that cooperate with the fasteners on the table. In one embodiment, the sides of the panels 16 and 16′ that face away from the table also have fasteners, which may be substantially similar to the fasteners that are mounted to the table. Thus, while the panels 16 and 16′ rest over the fasteners on the table, the panels 16 and 16′ have fasteners that may be used by another structure, such as a table cover 97 with fastener 98, which may be parallel hooks and loops strips, only one of which (98) is visible in FIG. 10.

In another embodiment, shown in FIG. 4, the positions of the two track portions may be reversed. In the embodiment of FIG. 4, the track portions 110 and 110′ are mounted to the bands 128 and 128′. The track portions 110 and 110′ are substantially similar in structure to the track portions 10 and 10′ in FIGS. 1-3, and therefore each of the track portions 110 and 110′ has a longitudinal channel. The channels may face inwardly toward a longitudinal centerline. The track portions 112 and 112′ are substantially similar to the track portions 12 and 12′, and contain cylindrical rods 130 and 130′ (rod 130′ is not visible in FIG. 4, but is identical to the rod 130) that are inserted into the channels of the track portions 110 and 110′.

The rods 130 and 130′ may be mounted at opposite edges to a flexible band 114 that extends through a gap between the table 140 and a lower support 150 for the table 140. The rods 130 and 130′ are positioned at opposite edges of the table 140 so the channels in the track portions 110 and 110′ may receive the rods. The bands 128 and 128′ are thus mounted through the track portions 110 and 110′ to the track portions 112 and 112′ that are mounted to the table 140 via fixing of the band 114 thereto. By longitudinal relative movement the bands 128 and 128′ may be adjusted relative to, or even removed from, the track portions 112 and 112′ that are mounted to the table 140. This configuration thus permits longitudinal adjustment of the fastening bands 128 and 128′ relative to the band 114 that is mounted in the table 140.

The track portions may be attached to the table or the bands that extend over the patient using any conventional means. Interchangeable hook and loop attachment flaps may adapt to existing fastener adhesive provided with OEM table pads. The track portions thus may be easily removed and cleaned, discarded or stored when desired by removing the components that are fastened to the table. This is important because during procedures patient fluids may contact the table. To prevent cross-contamination between patients, the table area and the track need to be thoroughly cleaned between patients.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A fastening apparatus for an imaging device having a table upon which at least part of a patient may rest while an image is taken of the at least part of the patient, the apparatus comprising:
(a) a first track portion having spaced lips defining a channel with a first longitudinal axis, the lips separated at their terminal ends to define an elongated slot;
(b) a second track portion having a panel attached to a rod, the rod having a second longitudinal axis and a size that permits the rod to be disposed in the channel of the first track portion with the first and second longitudinal axes substantially parallel and the panel disposed in the slot, whereby the first track portion is longitudinally movable relative to the second track portion;
wherein one of the first and second track portions is configured for attachment to the table and another of the first and second track portions is configured for attachment to a first band that may extend around the at least part of the patient.

2. The fastening apparatus in accordance with claim 1, further comprising:
(a) a third track portion having spaced lips defining a channel therebetween having a third longitudinal axis, the lips separated at their terminal ends to define a slot;
(b) a fourth track portion having a panel attached to a rod, the rod having a fourth longitudinal axis and a size that permits the rod to be disposed in the channel of the third track portion with the third and fourth longitudinal axes substantially parallel and the panel disposed in the slot, whereby the third track portion is longitudinally movable relative to the fourth track portion;
wherein one of the third and fourth track portions is configured for attachment to the table and another of the third and fourth track portions is configured for attachment to a second band that is attachable to the first band.

3. The fastening apparatus in accordance with claim 2, wherein the first track portion is configured for attachment to a first side of the table, the third track portion is configured for attachment to a second, opposite lateral side of the table, the second track portion is configured for attachment to the first band and the fourth track portion is configured for attachment to the second band.

4. The fastening apparatus in accordance with claim 2, wherein the second track portion is configured for attachment to a first side of the table, the fourth track portion is configured for attachment to a second, opposite lateral side of the table, the first track portion is configured for attachment to the first band and the third track portion is configured for attachment to the second band.

5. The fastening apparatus in accordance with claim 2, wherein the first, second, third and fourth track portions are radiolucent.

6. An imaging apparatus having a table upon which at least part of a patient rests while an image is taken of the at least part of the patient, the imaging apparatus comprising:
(a) a first track portion having spaced lips defining a channel with a first longitudinal axis, the lips separated at their terminal ends to define an elongated slot;
(b) a second track portion having a panel attached to a rod with a second longitudinal axis, the rod being disposed in the channel of the first track portion with the first and second longitudinal axes substantially parallel and the panel disposed in the slot, whereby the first track portion is longitudinally movable relative to the second track portion;
wherein one of the first and second track portions is mounted to the table and another of the first and second track portions is mounted to a first band that extends at least partially across the at least part of the patient while the image is taken.

7. The imaging apparatus in accordance with claim 6, further comprising:
(a) a third track portion having spaced lips defining a channel with a third longitudinal axis, the lips separated at their terminal ends to define an elongated slot;
(b) a fourth track portion having a panel attached to a rod with a fourth longitudinal axis, the rod being disposed in the channel of the first track portion with the third and fourth longitudinal axes substantially parallel and the panel disposed in the slot, whereby the third track portion is longitudinally movable relative to the fourth track portion;
wherein one of the third and fourth track portions is mounted to the table and another of the third and fourth track portions is mounted to a second band that is attached to the first band.

8. The fastening apparatus in accordance with claim 7, wherein the first track portion is mounted to a first side of the table, the third track portion is mounted to a second, opposite lateral side of the table, the second track portion is mounted to the first band and the fourth track portion is mounted to the second band.

9. The fastening apparatus in accordance with claim 7, wherein the second track portion is mounted to a first side of the table, the fourth track portion is mounted to a second, opposite lateral side of the table, the first track portion is mounted to the first band and the third track portion is mounted to the second band.

10. The fastening apparatus in accordance with claim 7, wherein the first, second, third and fourth track portions are radiolucent.

11. A method of taking an image of at least part of a patient while the at least part of the patient rests upon a table, the method comprising:
(a) extending a first band over the at least part of the patient, the first band mounted at one end to one of first and second track portions and another of the first and second track portions is mounted to the table, wherein:
(i) the first track portion has spaced lips defining a channel with a first longitudinal axis, the lips separated at their terminal ends to define an elongated slot; and
(ii) the second track portion has a panel attached to a rod with a second longitudinal axis, the rod is disposed in the channel of the first track portion with the first and second longitudinal axes substantially parallel and the panel disposed in the slot;
(b) tightening the first band around the at least part of the patient by applying to the first band a tensile force that is transverse to the first and second longitudinal axes; and
(c) displacing at least one of the track portions along the first and second longitudinal axes relative to the other of the track portions while the first band extends over the at least part of the patient and the transverse tensile force is applied to the first band.

12. The method in accordance with claim 9, further comprising:
(a) attaching a second band to the first band, the second band mounted at one end to one of third and fourth track portions and another of the third and fourth track portions is mounted to the table, wherein:
(i) the third track portion has spaced lips defining a channel with a third longitudinal axis, the lips separated at their terminal ends to define an elongated slot; and
(ii) the fourth track portion has a panel attached to a rod with a fourth longitudinal axis, the rod is disposed in the channel of the third track portion with the third and fourth longitudinal axes substantially parallel and the panel disposed in the slot; and
(b) applying to the second band a tensile force that is transverse to the third and fourth longitudinal axes.
